(19) United States
(12) Patent Application Publication
(10) Pub. No.: US 2024/0194092 A1
(43) Pub. Date: Jun. 13, 2024

(54) Physical Model For Aiding In Surgical Plan Accuracy Assessment

(71) Applicant: MAKO Surgical Corp., Weston, FL (US)

(72) Inventor: Jonathan Mark Morgan, Biscayne Park, FL (US)

(73) Assignee: MAKO Surgical Corp., Weston, FL (US)

(21) Appl. No.: 18/528,839

(22) Filed: Dec. 5, 2023

Related U.S. Application Data
(60) Provisional application No. 63/431,905, filed on Dec. 12, 2022.

(51) Int. Cl. G09B 23/34; A61B 34/10

(52) U.S. Cl. CPC G09B 23/34; A61B 34/10; A61B 2034/108

(57) ABSTRACT

A physical model configured to be physically altered by a surgical instrument for providing feedback related to a surgical plan specific to an anatomy of a patient. The physical model has a body including a physical volume representative of the anatomy of the patient and a geometrical feature embedded within the physical volume wherein the geometrical feature is visually distinct from a remainder of the physical volume and wherein the geometrical feature has parameters that are based on the surgical plan. The physical volume is configured to be at least partially removed by the surgical instrument such that the geometrical feature is configured to be exposed for providing visual feedback about an accuracy of the surgical plan and/or an accuracy of the surgical instrument in carrying out the surgical plan.

### CROSS-REFERENCE TO RELATED APPLICATIONS

The subject patent application claims priority to and all benefits of U.S. Provisional Patent App. No. 63/431,905, filed Dec. 12, 2022, the entire contents of which are hereby incorporated by reference.

### BACKGROUND

Despite the ever-increasing advancement of surgical technology, it remains important for surgeons to maintain a high degree of confidence in the systems used to plan and execute a surgical procedure. Surgeon confidence before the surgery is just as important as surgeon confidence during the surgery. Before surgery, a surgeon typically creates and reviews a surgical plan. For joint arthroplasty procedures, the patient anatomy, e.g., bone, is usually manipulated in preparation for receiving a joint-replacement implant. The surgical plan can include the size and location of the implant relative to the bone. Once the plan is formalized, the surgery is performed according to the plan.

Due to a variety of factors, intraoperative corrections to the pre-operative surgical plan are often made by the surgeon. For instance, the surgeon may realize that the implant size or location needs adjustment, the amount of bone removed needs to be modified, or the type, approach, or use of a tool requires change. In any event, intraoperative corrections to the plan are generally undesirable because the surgeon must make an immediate decision about the best course of action while the patient is under anesthesia. Intraoperative corrections also indicate that either the preoperative surgical plan was sub-optimal and/or the execution according to the surgical plan was sub-optimal.

Many systems provide intraoperative feedback about the surgeon's execution relative to the surgical plan. One such system is described in U.S. Pat. No. 8,010,180 to Mako Surgical Corp., which provides a navigation display in the operating room to show a virtual model of the anatomy incorporating the surgical plan. As the surgeon removes material from the patient anatomy, in real time, the display shows a corresponding interactive tool removing virtual material from the virtual model of the bone. Different colors can be indicated relative to the virtual model depending on the actual cutting depth as compared with the preferred depth according to the surgical plan to provide the surgeon with intraoperative feedback. Intraoperative feedback remains an important feature of accurate surgery. However, the described intraoperative feedback obviously is not accessible outside of the surgery. Intraoperative feedback comes at the cost of the extensive surgical setup (e.g., navigation systems, robotic manipulators, surgical drapes, surgical tools, implant kits, etc.) and involvement of numerous staff and technicians. Therefore, intraoperative feedback alone does not address establishing surgeon confidence in the surgical plan, or execution of the surgical plan, before the surgery.

Other systems provide a preoperative surgical simulator wherein the surgeon virtually performs a surgery in a virtual operating room. Although this technique may help the surgeon get acquainted with the planned surgery in a preoperative sense, virtual simulations fail to address providing the surgeon with “real” feedback about the surgery. Virtual simulations cannot adequately replicate the human senses involved with physically using a tool to perform a planned surgery. While virtual simulations may be adequate for purposes such as training, virtual simulations fall short of providing the surgeon with immediate, tangible (physical) feedback about the accuracy of the surgical plan and/or accuracy in carrying out the surgical plan. In other words, virtual simulations do little to establish surgeon confidence in the surgical plan, or execution of the surgical plan, before the surgery.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, a physical surgical planning aide is provided, comprising: a physical volume and a geometrical feature embedded within the physical volume wherein the geometrical feature is different from a remainder of the physical volume and wherein the geometrical feature has parameters that are based on the surgical plan.

According to a second aspect, a physical model configured to be physically altered by a surgical instrument for providing feedback related to a surgical plan specific to an anatomy of a patient is provided. The physical model includes a body including a physical volume and a geometrical feature embedded within the physical volume wherein the geometrical feature is visually distinct from a remainder of the physical volume and wherein the geometrical feature has parameters that are based on the surgical plan. Further, the physical volume is configured to be at least partially removed by the surgical instrument such that the geometrical feature is configured to be exposed for providing visual feedback about an accuracy of the surgical plan and/or an accuracy of the surgical instrument in carrying out the surgical plan.

According to a third aspect, a surgical planning system including a surgical instrument and a physical model is provided. The physical model is configured to be physically altered by the surgical instrument to provide feedback related to a surgical plan specific to an anatomy of a patient. The physical model includes a body. The body includes a physical volume and a geometrical feature embedded within the physical volume. The geometrical feature is visually distinct from a remainder of the physical volume and the has parameters that are based on the surgical plan. The physical volume is configured to be at least partially removed by the surgical instrument such that the geometrical feature is exposed to provide visual feedback about an accuracy of the surgical plan and/or an accuracy of the surgical instrument in carrying out the surgical plan.

According to a fourth aspect, a method of utilizing a surgical planning system for providing feedback related to a surgical plan specific to an anatomy of a patient is provided. The surgical planning system includes a surgical instrument and a physical model configured to be physically altered by the surgical instrument. The physical model includes a body including a physical volume. The physical volume includes a geometrical feature embedded within the physical volume, which is visually distinct from a remainder of the physical volume and has parameters that are based on the surgical plan. The method includes utilizing the surgical instrument for at least partially removing the physical volume and for exposing the geometrical feature, and providing, based on the exposed geometrical feature, visual feedback about an accuracy of the surgical plan and/or an accuracy of the surgical instrument in carrying out the surgical plan.

According to a fifth aspect, a method of producing a physical model configured to be physically altered by a surgical instrument for providing feedback related to a surgical plan specific to an anatomy of a patient is provided. The physical model includes a body including a physical volume. The physical volume includes a geometrical feature embedded within the physical volume. The geometrical feature is visually distinct from a remainder of the physical volume and has parameters that are based on the surgical plan. The method includes obtaining, with a computing system, which may include a computer-aided design program, data related to the surgical plan specific to the anatomy of the patient; evaluating, with the computing system, the data related to the surgical plan for identifying or determining a shape of the physical model and the parameters of the geometrical feature embedded within the physical volume of the physical model; and thereafter commanding, with the computing system, a machine for additively manufacturing the shape of the physical model and the geometrical feature embedded within the physical volume of the physical model.

According to a sixth aspect, a modular system including a physical model and a base is provided. The modular system is configured to be physically altered by a surgical instrument for providing feedback related to a surgical plan specific to an anatomy of a patient. The physical model includes a body and a first mounting interface coupled to the body. The body includes a physical volume and a geometrical feature embedded within the physical volume. The geometrical feature is visually distinct from a remainder of the physical volume and has parameters that are based on the surgical plan. The base includes a second mounting interface arranged to couple with the first mounting interface of the physical model such that the physical model is configured to be detachably coupled to the base. The physical volume is configured to be at least partially removed by the surgical instrument such that the geometrical feature is exposed to provide visual feedback about an accuracy of the surgical plan and/or an accuracy of the surgical instrument in carrying out the surgical plan.

According to a seventh aspect, a modular system including a physical model is provides. The modular system is configured to be physically altered by a surgical instrument for providing feedback related to a surgical plan specific to an anatomy of a patient. The physical model includes a body. The body includes a physical volume. The physical volume includes a first sub-volume and a second sub-volume. The second sub-volume includes a geometrical feature corresponding to a surgical plan embedded within the second sub-volume. The second sub-volume is configured to be attached to the first sub-volume and at least partially removed by the surgical instrument such that the geometrical feature is exposed to provide visual feedback about an accuracy of the surgical plan and/or an accuracy of the surgical instrument in carrying out the surgical plan.

Any of the above aspects can be combined in part or in whole with any other aspect. Any of the above aspects, whether combined in part or in whole, can be further combined with any of the following implementations, in full or in part.

In some implementations, to be visually distinct from the remainder of the physical volume, the geometrical feature includes a material property that is different from a material property of the remainder of the physical volume.

In some implementations, the physical volume and geometrical feature are formed by additive manufacturing. In some implementations, the physical volume is formed with regions of variable density correlating to density information of the anatomy based on the surgical plan.

In some implementations, the geometrical feature comprises a boundary surface embedded within the physical volume. In some implementations, the boundary surface is indicative of a region of the anatomy that should be avoided by the surgical instrument according to the surgical plan. In some implementations, the boundary surface is indicative of a planned resection surface of the anatomy that is configured to receive an implant according to the surgical plan, and wherein the planned resection surface has parameters of shape, size and position based on the implant of the surgical plan and a geometry of the anatomy.

In some implementations, the physical volume is shaped to correspond to the anatomy of the patient or shaped according to a generic geometry. In some implementations, the physical volume is representative of a distal femur of the patient and the planned resection surface comprises a plurality of connected planar surfaces indicative of target surfaces of the distal femur that are configured to receive a femoral implant. In some implementations, the physical volume is representative of a tibia of the patient and the planned resection surface comprises a planar surface indicative of a target surface of the tibia that is configured to receive a tibial implant. In some implementations, the physical volume is representative of a pelvis and acetabulum of the patient and the planned resection surface comprises a concave surface indicative of a target surface of the acetabulum that is configured to receive an acetabular cup implant. In some implementations, the physical volume is representative of a scapula and a glenoid of the patient and the planned resection surface comprises a concave surface indicative of a target surface of the glenoid that is configured to receive a glenoid implant.

In some implementations, a depth indicator having variable cross-sections is embedded within a thickness of the boundary surface, and wherein the boundary surface and the depth indicator are configured to be at least partially removed by the surgical instrument such that a cross-section of the depth indicator is configured to be exposed to provide visual feedback related to the thickness of the boundary surface removed or remaining

In some implementations, the geometrical feature comprises a first boundary surface, a second boundary surface, and a third boundary surface, wherein the first boundary surface is stacked on top of the second boundary surface, and the second boundary surface is stacked on top of the third boundary surface, and wherein each boundary surface is visually distinct from the other boundary surfaces. In some implementations, the first, second, and third boundary surfaces are configured to be exposed to provide visual feedback related to a cutting accuracy of the surgical instrument. In some implementations, the first boundary surface is configured to be exposed to indicate an undercut, the second boundary surface is configured to be exposed to indicate an accurate cut, and the third boundary surface is configured to be exposed to indicate an overcut.

In some implementations, the geometrical feature comprises a sub-volume embedded within and surrounded by the physical volume. In some implementations, the sub-volume is indicative of a planned resection volume of the anatomy that is configured to receive an implant according to the surgical plan, and wherein the planned resection volume has parameters of shape, size and position based on the implant of the surgical plan and a geometry of the anatomy.

In some implementations, the physical volume is representative of a proximal femur of the patient and the planned resection volume comprises a geometry indicative of a target volume of the proximal femur that is configured to receive a femoral stem implant. In some implementations, the physical volume is representative of a scapula and a glenoid of the patient and the planned resection volume comprises a geometry indicative of a target volume of the glenoid that is configured to receive a glenoid implant. In some implementations, the physical volume is representative of a proximal humerus of the patient and the planned resection volume comprises a geometry indicative of a target volume of the proximal humerus that is configured to receive a humeral stem implant. In some implementations, the physical volume is representative of a vertebra of the patient and the planned resection volume comprises a geometry indicative of a target volume of the vertebra that is configured to receive a pedicle screw. In some implementations, the physical volume is representative of a distal femur of the patient and the planned resection volume comprises a geometry indicative of a target volume of the distal femur that is configured to receive a peg of a femoral implant. In some implementations, the physical volume is representative of a tibia of the patient and the planned resection volume comprises a geometry indicative of a target volume of the tibia that is configured to receive a stem of a tibial implant.

In some implementations, a depth indicator having variable cross-sections is embedded within a thickness of the sub-volume, and wherein the sub-volume and the depth indicator are configured to be at least partially removed by the surgical instrument such that a cross-section of the depth indicator is configured to be exposed to provide visual feedback related to the thickness of the sub-volume removed or remaining.

In some implementations, the geometrical feature comprises a first sub-volume boundary surface, a second sub-volume boundary surface, and a third sub-volume boundary surface, wherein the first sub-volume boundary surface is surrounded by the second sub-volume boundary surface, and the second sub-volume boundary surface is surrounded by the third sub-volume boundary surface, and wherein each sub-volume boundary surface is visually distinct from the other sub-volume boundary surfaces. In some implementations, the first, second, and third sub-volume boundary surfaces are configured to be exposed to provide visual feedback related to a cutting accuracy of the surgical instrument. In some implementations, the first sub-volume boundary surface is configured to be exposed to indicate an undercut, the second sub-volume boundary surface is configured to be exposed to indicate an accurate cut, and the third sub-volume boundary surface is configured to be exposed to indicate an overcut.

In some implementations, the geometrical feature comprises a path embedded within the physical volume. In some implementations, the path is indicative of a planned path of the surgical instrument relative to the anatomy according to the surgical plan, and wherein the planned path has parameters of shape, length and position based on the surgical plan and a geometry of the anatomy. In some implementations, the physical volume is representative of a bone of the patient and the path is indicative of the planned path of the surgical instrument designed to remove a portion from the bone. In some implementations, the planned path of the surgical instrument designed to install an implant in the bone. In some implementations, the geometrical feature comprises a first path formed as a first cylinder and a second path formed as a second cylinder surrounding the first cylinder, wherein the first path is visually distinct from the second path. In some implementations, the first and second paths are configured to be exposed to provide visual feedback related to a path accuracy of the surgical instrument. In some implementations, the first path is configured to be exposed to indicate an accurate path of the surgical instrument, and the second path is configured to be exposed to indicate an inaccurate path of the surgical instrument.

In some implementations, the physical body is configured to couple to a base. In some implementations, the body includes a first mounting interface, and the base includes a second mounting interface arranged to couple with the first mounting interface such that the body is configured to be detachably coupled to the base. In some implementations, the body is configured to be removed from the base after the physical volume has been at least partially removed by the surgical instrument. In some implementations, a replacement body is configured to be coupled to the base. In some implementations, the replacement body is substantially similar to the body.

In some implementations, the physical volume includes a first sub-volume and a second sub-volume. In some implementations, the second sub-volume includes a geometrical feature corresponding to a surgical plan embedded within the second sub-volume. In some implementations, the second sub-volume is configured to be attached to the first sub-volume and at least partially removed by the surgical instrument such that the geometrical feature is exposed to provide visual feedback about an accuracy of the surgical plan and/or an accuracy of the surgical instrument in carrying out the surgical plan. In some implementations, the second sub-volume is configured to be detached from the first sub-volume after the second sub-volume has been at least partially removed by the surgical instrument to facilitate replacement of the second sub-volume.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of one implementation of a surgical system comprising a robotic manipulator and a navigation system including a localization device.

FIG. 2 is a block diagram of a control system for controlling the surgical system, according to one implementation.

FIG. 3 is a perspective view of a physical model including a geometrical feature according to one implementation.

FIG. 4 is a perspective view of a physical model including a geometrical feature according to another implementation.

FIG. 5 is a perspective view of a modular physical model including a geometrical feature according to another implementation.

FIG. 6 is a partial, cross-sectional view, of the geometrical feature of FIG. 3.

FIG. 7 is a perspective view of the physical model with a portion of the geometrical feature of FIG. 3 exposed.

FIG. 8 is a perspective view of the physical model of FIG. 3 with exemplary portions of the geometrical feature exposed by a surgical saw.

FIG. 9 is a perspective view of the physical model of FIG. 3 with exemplary portions of the geometrical feature exposed by a surgical bur.

FIG. 10A is a perspective view of a physical model with a geometrical feature according to another implementation.

FIG. 10B is a cross-sectional view of the physical model of FIG. 10A.

FIG. 11 is a cross-sectional view of the physical model and geometrical feature of FIG. 10A after being altered by a surgical instrument to include an accurately bored hole.

FIG. 12 is a cross-sectional view of the physical model and geometrical feature of FIG. 10A after being altered by a surgical instrument to include an inaccurately bored hole according to one implementation.

FIG. 13 is a cross-sectional view of the physical model and geometrical feature of FIG. 10A after being altered by a surgical instrument to include an inaccurately bored hole according to another implementation.

FIG. 14 is a cross-sectional view of the physical model and geometrical feature of FIG. 10A after being altered by a surgical instrument to include an inaccurately bored hole according to yet another implementation.

FIG. 15 is a cross-sectional view of the physical model and geometrical feature of FIG. 10A after being altered by a surgical instrument to include an inaccurately bored hole according to yet another implementation.

### DETAILED DESCRIPTION

### I. Example System Overview

Referring to the Figures, wherein like numerals indicate like or corresponding parts throughout the several views, a surgical robotic system (hereinafter “system”) 10, a physical model 200, and methods for using the same are shown throughout.

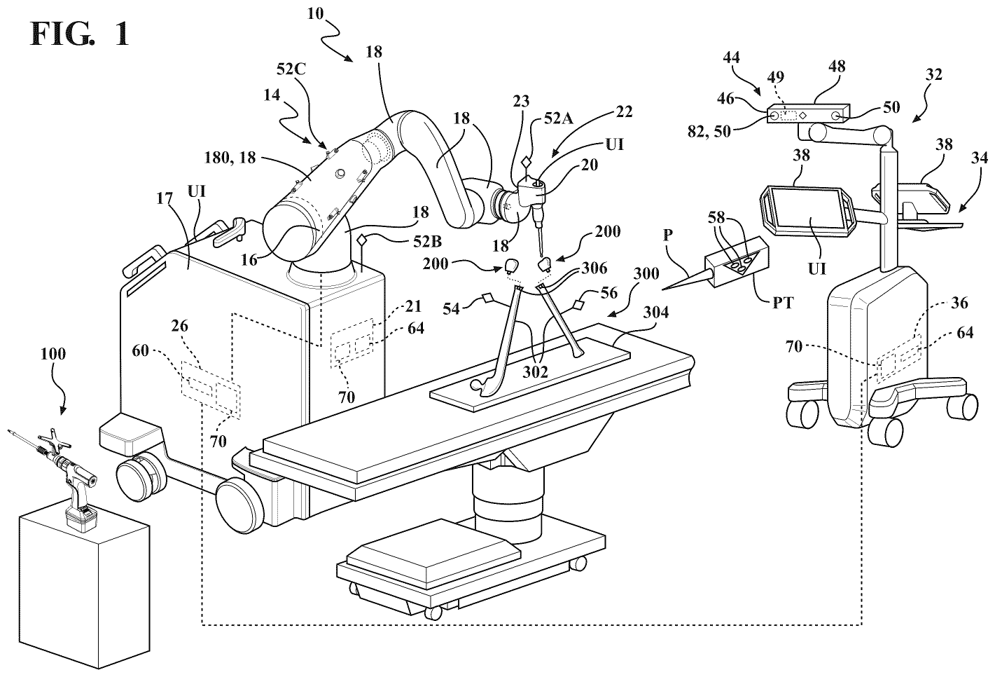

Referring to FIG. 1, a surgical robotic system 10 is illustrated. The system 10 is useful for treating a surgical site or anatomical volume (A) of a patient, such as treating bone or soft tissue. In FIG. 1, the patient is omitted and has otherwise been replace by a model femur and a model tibia for reasons that will become clear based on the description below. The surgical procedure may involve tissue removal or other forms of treatment. Treatment may include cutting, coagulating, lesioning the tissue, other in-situ tissue treatments, or the like. In some examples, the surgical procedure involves partial or total knee or hip replacement surgery, shoulder replacement surgery, spine surgery, or ankle surgery. In some examples, the system 10 is designed to cut away material to be replaced by surgical implants, such as hip and knee implants, including unicompartmental, bicompartmental, multicompartmental, or total knee implants. Some of these types of implants are shown in U.S. Patent Application Publication No. 2012/0330429, entitled, “Prosthetic Implant and Method of Implantation,” the disclosure of which is hereby incorporated by reference. The system 10 and techniques disclosed herein may be used to perform other procedures, surgical or non-surgical. The system 10 may be used in industrial (non-surgical) applications or other applications where robotic systems are utilized.

The system 10 includes a (robotic) manipulator 14. The manipulator 14 has a base 16 and plurality of links 18. A manipulator cart 17 supports the manipulator 14 such that the manipulator 14 is supported by the manipulator cart 17. The links 18 collectively form one or more arms of the manipulator 14. In some implementations, and as further described below, one or more of the links 18 is a trackable link 180 and includes tracking elements such as LEDs and photosensors. The manipulator 14 may have a serial arm configuration (as shown in FIG. 1), a parallel arm configuration, or any other suitable manipulator configuration. In other examples, more than one manipulator 14 may be utilized in a multiple arm configuration. In one example, the manipulator 14 can have a configuration such as the robotic manipulator described in U.S. Pat. No. 10,327,849, entitled “Robotic System and Method for Backdriving the Same”, the contents of which are hereby incorporated by reference in its entirety.

The base 16 of the manipulator 14 is generally a portion of the manipulator 14 that provides a fixed reference coordinate system for other components of the manipulator 14 or the system 10 in general. Generally, the origin of a manipulator coordinate system MNPL is defined at the fixed reference of the base 16. The base 16 may be defined with respect to any suitable portion of the manipulator 14, such as one or more of the links 18. Alternatively, or additionally, the base 16 may be defined with respect to the manipulator cart 17, such as where the manipulator 14 is physically attached to the manipulator cart 17. In other examples, the manipulator 14 can be a hand-held manipulator where the base 16 is a base portion of a tool (e.g., a portion held free-hand by a user) and the tool tip is movable relative to the base portion. The base portion has a reference coordinate system that is tracked and the tool tip has a tool tip coordinate system that is computed relative to the reference coordinate system (e.g., via motor and/or joint encoders and forward kinematic calculations). Movement of the tool tip can be controlled to follow the path since its pose relative to the path can be determined.

The manipulator 14 and/or manipulator cart 17 house a manipulator controller 26, or other type of control unit. The manipulator controller 26 may comprise one or more computers, or any other suitable form of controller that directs the motion of the manipulator 14. The manipulator controller 26 may have a central processing unit (CPU) and/or other processors, memory, and storage. The manipulator controller 26 is loaded with software as described below. The processors could include one or more processors to control operation of the manipulator 14. The processors can be any type of microprocessor, multi-processor, and/or multi-core processing system. The manipulator controller 26 may additionally, or alternatively, comprise one or more microcontrollers, field programmable gate arrays, systems on a chip, discrete circuitry, and/or other suitable hardware, software, or firmware that is capable of conducting the functions described herein. The term processor is not intended to limit any implementation to a single processor. The manipulator 14 may also comprise a user interface UI with one or more displays and/or input devices (e.g., push buttons, keyboard, mouse, microphone (voice-activation), gesture control devices, touchscreens, etc.).

A tool 20 couples to the manipulator 14 and is movable relative to the base 16 to interact with the anatomy in certain modes. The tool 20 is a physical and surgical tool and is or forms part of an end effector 22 supported by the manipulator 14 in certain implementations. More specifically, the manipulator 14 may include a first mounting interface configured to removably receive the end effector 22. In order to secure to the first mounting interface, the end effector 22 may include an end effector body 23 which includes a second mounting interface configured to couple to the first mounting interface. The tool 20 may be grasped by the user. One possible arrangement of the manipulator 14 and the tool 20 is described in U.S. Pat. No. 9,119,655, entitled, “Surgical Manipulator Capable of Controlling a Surgical Instrument in Multiple Modes,” the disclosure of which is hereby incorporated by reference. The manipulator 14 and the tool 20 may be arranged in alternative configurations. The tool 20 can be like that shown in U.S. Pat. No. 9,566,121, filed on Mar. 15, 2014, entitled, “End Effector of a Surgical Robotic Manipulator,” hereby incorporated by reference.

The tool 20 may comprise a tool controller 21 to control operation of the tool 20, such as to control power to the tool (e.g., to a rotary motor of the tool 20), control movement of the tool 20, control irrigation/aspiration of the tool 20, and/or the like. The tool controller 21 may be in communication with the manipulator controller 26 or other components. The tool 20 may also comprise a user interface UI with one or more displays and/or input devices (e.g., push buttons, keyboard, mouse, microphone (voice-activation), gesture control devices, touchscreens, etc.). The manipulator controller 26 controls a state (position and/or orientation) of the tool 20 (e.g., the TCP) with respect to a coordinate system, such as the manipulator coordinate system MNPL. The manipulator controller 26 can control (linear or angular) velocity, acceleration, or other derivatives of motion of the tool 20.

The system 10 further includes a navigation system 32. One example of the navigation system 32 is described in U.S. Pat. No. 9,008,757, entitled, “Navigation System Including Optical and Non-Optical Sensors,” hereby incorporated by reference. The navigation system 32 tracks movement of various objects. Such objects include, for example, the manipulator 14, the tool 20 and the anatomy, e.g., femur F and tibia T. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformations.

The navigation system 32 includes a cart assembly 34 that houses a navigation controller 36, and/or other types of control units. A navigation user interface UI is in operative communication with the navigation controller 36. The navigation user interface includes one or more displays 38. The navigation system 32 is capable of displaying a graphical representation of the relative states of the tracked objects to the user using the one or more displays 38. The navigation user interface UI further comprises one or more input devices to input information into the navigation controller 36 or otherwise to select/control certain aspects of the navigation controller 36. Such input devices include interactive touchscreen displays. However, the input devices may include any one or more of push buttons, a keyboard, a mouse, a microphone (voice-activation), gesture control devices, and the like.

The navigation system 32 also includes a navigation localizer 44 coupled to the navigation controller 36. The relative location of the localizer 44 with respect to the manipulator 14 in FIG. 1 is provided only for illustrative purposes to show the respective components and is not necessarily representative of the optimal manner in which to setup the localizer 44. In one example, the localizer 44 is an optical localizer and includes a camera unit 46. The camera unit 46 has an outer casing 48 that houses one or more optical sensors 50. The localizer 44 may comprise its own localizer controller 49 and may further comprise a video camera VC. The localization device 44 may include an IR transmitter 82 configured to send and receive infrared (IR) signals. The IR transmitter 82 is in communication with the localizer controller 49 such that signals received by the IR transmitter 82 can be relayed to the localizer controller 49. The IR transmitter 82 may be in communication with the various trackers utilized by the surgical robotic system 10. Any IR communications from the localizer 44 may originate from the IR transmitter 82.

The navigation system 32 includes one or more trackers. In one example, the trackers include a pointer tracker PT, one or more robotic or tool trackers 52A, 52B, 52C a first patient tracker 54, and a second patient tracker 56. The first patient tracker 54 is firmly affixed to the femur F of the patient, and the second patient tracker 56 is firmly affixed to the tibia T of the patient. In this example, the patient trackers 54, 56 are firmly affixed to sections of bone. The pointer tracker PT is firmly affixed to a pointer P used for registering the anatomy to the localizer coordinate system LCLZ.

The tracker 52A, herein referred to as an end effector tracker 52A, may be secured to any part of the end effector 22. For example, the end effector tracker 52A may be secured to the end effector body 23 or the tool 20. In addition, the end effector tracker 52A may be integrated into the end effector 22 or one of the mounting interfaces. For example, the end effector tracker 52A may comprise one tracking element (e.g., light emitting diode) or a plurality of tracking elements integrated into or coupled to the end effector body 23. The tracking elements may be arranged in an EE tracking geometry such that the localizer 44 can differentiate the end effector tracker 52A from the other tracker 52B, 52C, 54, 56, PT based on the EE tracking geometry. The end effector tracker 52A may further include a sensor (e.g., a photosensor) configured to receive signals from the localizer 44 such that the localizer 44 can control the end effector tracker 52A.

The tracker 52B, herein referred to as a base tracker 52B, may be movably and/or stowably secured to the base 16. For example, the base 16 may further include an adjustable arm configured to support the base tracker 52B. The adjustable arm may include a tracker interface configured to couple to the base tracker 52B. The adjustable arm may be pivotably secured to the base 16 at a connection point such that the adjustable arm may be moved between a stowed position and various deployed positions. The adjustable arm may be considered to be in the stowed position when it is folded flat up against the base 16, and the adjustable arm may be considered to be in one of the deployed positions when it is pivoted about the connection point so as to form an angle with the side of the base 16. Such as arrangement allows the base tracker 52B to be coupled to the adjustable arm at the tracker interface and moved relative to the base 16 until the tracker 52B is in a desired position. The base tracker 52B may further include a sensor (e.g., a photosensor) configured to receive signals from the localizer 44 such that the localizer 44 can control the base tracker 52B.

The tracker 52C, herein referred to as a link tracker 52C, may be coupled to one of the links 18. The link 18 including the tracker 52C is realized as the trackable link 180. An example of the link tracker 52C and the trackable link 180 is described in U.S. Pat. App. No. 63/315,665, entitled “Robotic System Including a Link Tracker,” the entirety of which is hereby incorporated by reference.

The localizer 44 may need to initialize the trackers 52A, 52B, 52C at the request of the user, procedure, or the navigation system 32. Alternatively, any one or more of the trackers 52A, 52B, 52C may comprise controllers to recognize, based on signals, or in response to any other condition, when the respective tracker 52 should be initialized. In other examples, any of the trackers 52 may be active and ready so long as power is provided to the tracker. For instance, the end effector tracker 52A may only include one or more activated LEDs and not have a component or a controller configured to receive signals from the localizer 44. Since the end effector tracker 52A may not be able to receive communications from the localizer 44 in this example, the tracker 52A can be enabled at all times or otherwise controlled by the user via the various user interfaces UI.

Any one or more of the trackers 52A, 52B, 52C, 54, 56, PT may include active markers 58. The active markers 58 may include light emitting diodes (LEDs). The LEDs may be configured to provide tracking information to the navigation system 32, and the photosensors may be configured to receive signals from the navigation system 32. Alternatively, the trackers 52A, 52B, 52C, 54, 56, PT may have passive markers, such as reflectors, which reflect light emitted from the camera unit 46. In other examples, any or all of the trackers 52A, 52B, 52C, 54, 56, PT may utilize a combination of active and passive tracking elements. Other suitable markers not specifically described herein may be utilized. Any one or more of the trackers 52A, 52B, 52C, 54, 56, PT may include photosensors or infrared receivers to receive control signals from the navigation system 32.

The navigation controller 36 may comprise one or more computers, or any other suitable form of controller. The navigation controller 36 has a central processing unit (CPU) and/or other processors, memory (not shown), and storage (not shown). The processors can be any type of processor, microprocessor, or multi-processor system. The navigation controller 36 is loaded with software. The software, for example, converts the signals received from the localizer 44 into data representative of the position and orientation of the objects being tracked. The navigation controller 36 may additionally, or alternatively, comprise one or more microcontrollers, field programmable gate arrays, systems on a chip, discrete circuitry, and/or other suitable hardware, software, or firmware that is capable of conducting the functions described herein.

In another example, the navigation system 32 and/or localizer 44 are radio frequency (RF)-based. For example, the navigation system 32 may comprise an RF transceiver coupled to the navigation controller 36. The manipulator 14, the tool 20, and/or the patient may comprise RF emitters or transponders attached thereto. The RF emitters or transponders may be passive or actively energized. The RF transceiver transmits an RF tracking signal and generates state signals to the navigation controller 36 based on RF signals received from the RF emitters. The navigation controller 36 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers 52A, 52B, 52C, 54, 56, PT shown in FIG. 1.

In another example, the navigation system 32 and/or localizer 44 are electromagnetically based. For example, the navigation system 32 may comprise an EM transceiver coupled to the navigation controller 36. The manipulator 14, the tool 20, and/or the patient may comprise EM components attached thereto, such as any suitable magnetic tracker, electro-magnetic tracker, inductive tracker, or the like. The trackers may be passive or actively energized. The EM transceiver generates an EM field and generates state signals to the navigation controller 36 based upon EM signals received from the trackers. The navigation controller 36 may analyze the received EM signals to associate relative states thereto. Again, such navigation system 32 examples may have structural configurations that are different than the navigation system 32 configuration shown in FIG. 1.

In yet another example, the navigation system 32 and/or localizer 44 are machine vision/computer vision based. For example, the navigation system 32 may comprise a machine or computer vision camera coupled to the navigation controller 36. The manipulator 14, the tool 20, and/or the patient may comprise vision detectable elements attached thereto, such as any suitable pattern, color, barcode, QR code, or the like. The vision detectable elements may be passive or actively energized. The navigation controller 36 may analyze image and/or depth data from the vision detectable elements to associate relative states thereto. Again, such navigation system 32 examples may have structural configurations that are different than the navigation system 32 configuration shown in FIG. 1.

The navigation system 32 can use any combination of the above-described localization techniques. The navigation system 32 may have any other suitable components or structure not specifically recited herein. Furthermore, any of the techniques, methods, and/or components described above with respect to the navigation system 32 shown may be implemented or provided for any of the other examples of the navigation system 32 described herein.

As will be appreciated in tandem with section II below, a handheld surgical instrument 100 may be used instead of or in combination with the manipulator 14 of the surgical robotic system 10. For example, a surgeon may (1) direct the manipulator 14 to act on a physical model (introduced and described in detail below), (2) control the handheld surgical instrument 100 with their hand(s) to act on the physical model, and/or (3) use the manipulator 14 to control the handheld surgical instrument 100 to act on the physical model. The surgical instrument 100 may include a tracker to allow the navigation system 32 to detect and track the pose of the surgical instrument 100.

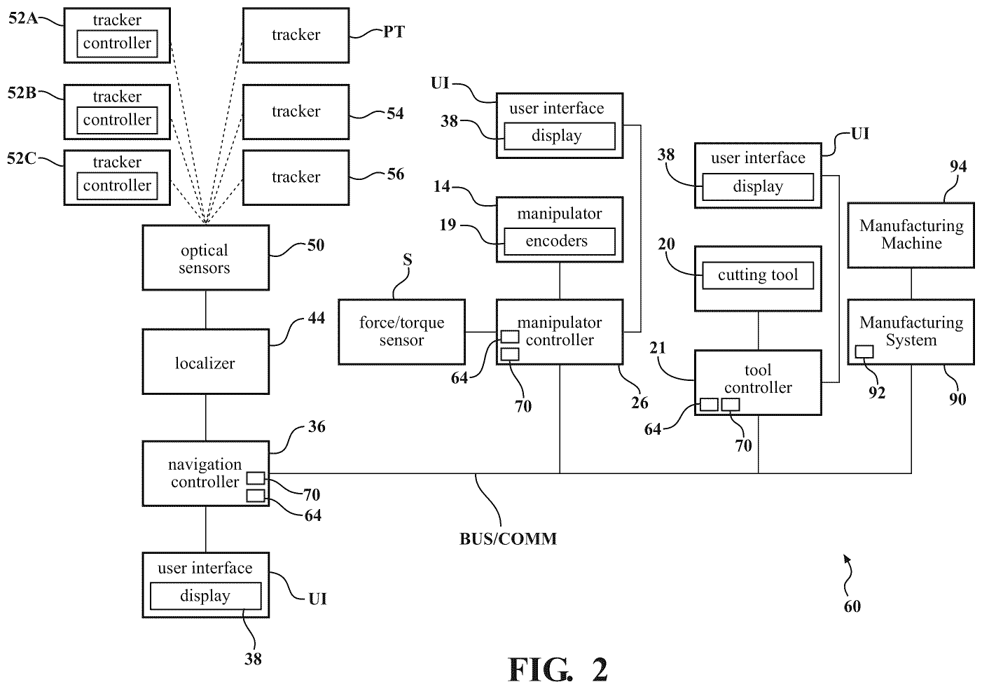

Referring to FIG. 2, the system 10 may include a control system 60 that comprises, among other components, the manipulator controller 26, the navigation controller 36, the tool controller 21, and a manufacturing system 90. The control system 60 may further include one or more software programs and software modules. The software modules may be part of the program or programs that operate on the manipulator controller 26, navigation controller 36, tool controller 21, or any combination thereof, to process data to assist with control of the system 10. The software programs and/or modules include computer readable instructions stored in non-transitory memory 64 on the manipulator controller 26, navigation controller 36, tool controller 21, or a combination thereof, to be executed by one or more processors 70 of the controllers 21, 26, 36. The memory 64 may be any suitable configuration of memory, such as RAM, non-volatile memory, etc., and may be implemented locally or from a remote database. Additionally, software modules for prompting and/or communicating with the user may form part of the program or programs and may include instructions stored in memory 64 on the manipulator controller 26, navigation controller 36, tool controller 21, or any combination thereof. The user may interact with any of the input devices of the navigation user interface UI or other user interface UI to communicate with the software modules. The user interface software may run on a separate device from the manipulator controller 26, navigation controller 36, and/or tool controller 21.

The control system 60 may comprise any suitable configuration of input, output, and processing devices suitable for conducting the functions and methods described herein. The control system 60 may comprise the manipulator controller 26, the navigation controller 36, or the tool controller 21, or any combination thereof, or may comprise only one of these controllers. These controllers may communicate via a wired bus or communication network as shown in FIG. 2, via wireless communication, or otherwise. The control system 60 may also be referred to as a controller. The control system 60 may comprise one or more microcontrollers, field programmable gate arrays, systems on a chip, discrete circuitry, sensors, displays, user interfaces, indicators, and/or other suitable hardware, software, or firmware that is capable of conducting the functions described herein.

The surgical robotic system 10 can be operated in a manual mode or an automated mode of operation. In the manual mode, the operator manually directs, and the manipulator 14 controls, movement of the tool 20 relative to the target. The operator physically contacts the tool 20 to cause movement of the tool 20. The manipulator 14 monitors the forces and torques placed on the tool 20 by the operator in order to position the tool 20. These forces and torques can be measured by a sensor that is part of the manipulator 14. In response to the applied forces and torques, the manipulator 14 mechanically moves the tool 20 in a manner that emulates the movement that would have occurred based on the forces and torques applied by the operator. Movement of the tool 20 in the manual mode can be constrained in relation to the virtual boundaries, which can delineate a region that the tool 20 is allowed to move as compared with a region into which the tool 20 is prohibited from moving. The region can be in space or can be located relative to a surgical object. The virtual boundaries can provide the operator with haptic feedback or force to indicate the location of the intermediate virtual boundary to the operator. For instance, by virtue of the manipulator 14 preventing or resisting movement of the tool 20 beyond the virtual boundary, the operator haptically senses a virtual wall when reaching the virtual boundary.

Additionally, or alternatively, the surgical robotic system 10 can be operated in an automated mode. In the automated mode, the manipulator 14 directs autonomous movement of the tool 20 relative to the target. The manipulator 14 is capable of moving the tool 20 free of operator assistance. Free of operator assistance may mean that an operator does not physically contact the tool 20 to apply force to move the tool 20. Instead, the operator may use some form of control to remotely manage starting and stopping of movement. For example, the operator may hold down a button of a remote control to start movement of the tool 20 and release the button to stop movement of the tool 20. Alternatively, the operator may press a button to start movement of the tool 20 and press a button to stop movement of the tool 20. Movement of the tool 20 in the automated mode may be constrained in relation to a predetermined path of movement and/or virtual boundary derived from a surgical plan.

The surgical robotic system 10 and methods of using the same may be like the surgical robotic system described in U.S. Pat. No. 9,812,035, entitled “System and Method for Demonstrating Planned Autonomous Manipulation of an Anatomy”, or like that described in U.S. Pat. No. 10,098,704, entitled “System and Method for Manipulating an Anatomy”, the entire contents of both of which are hereby incorporated by reference.

The surgical robotic system 10 may be utilized by an operator, such as the surgeon, in combination with the below-described physical model and to conduct methods involving the same. The surgical instrument 100 may also be used alone or in combination with the surgical robotic system 10 to act on the physical model and to conduct methods involving the same.

The control system 60 may further include the manufacturing system 90 to facilitate the manufacturing of various surgical components as further described in Section II below. The manufacturing system 90 is in communication with the rest of the control system 60 and may include a manufacturing controller 92. The manufacturing controller 92 may be configured to receive data from the navigation controller 36, the manipulator controller 26, the tool controller 21, and/or other elements of the control system 60. For example, the manufacturing controller 92 may receive patient imaging data, surgically defined annotations, target boundaries, target trajectories, planned tool paths, locations for any defined feature, thickness of any defined feature, density of volume(s), planned implant sizes and/or positions, and/or other data useful for manufacturing the various surgical components. In order to conduct the manufacturing, the manufacturing system may include a manufacturing machine 94. The manufacturing machine 94 may be an additive manufacturing system or any other suitable form of manufacturing system. According to one example method, the manufacturing controller 92 may receive data from other elements of the control system 60, develop a computer model based on the received data (e.g., a CAD model), and command the manufacturing machine 94 to manufacture a surgical component according to the computer model.

### II. Physical Model and Associated Methods

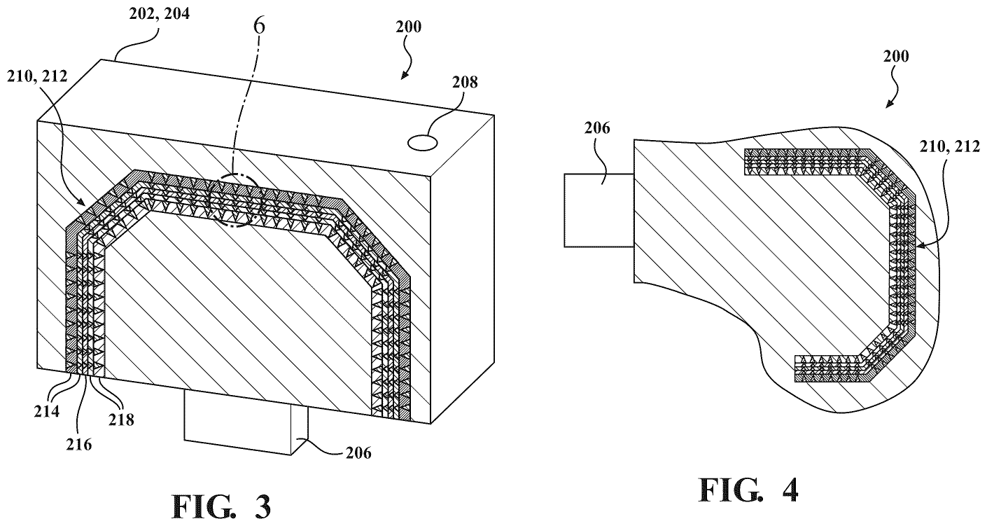

With reference to FIGS. 3 to 15 and described in this section are examples of physical models for aiding in surgical plan accuracy assessment. The techniques herein can be implemented using any implementation of the systems described above. Methods and software for implementing these techniques are also contemplated and described.

Referring to FIG. 3, one example of a physical model 200 is shown. The physical model 200 includes a body 202 including a physical volume 204, as well as one or more geometrical feature(s) 210 embedded within the physical volume 204. The physical volume 204 is bounded by an outer surface of the body 202. The physical volume 204 is configured to be at least partially removed by the surgical instrument 100 such that the geometrical feature 210 is exposed to provide visual feedback about an accuracy of the surgical plan and/or an accuracy of the surgical instrument in conducting the surgical plan. As described below, the shape of the body 202, volume 204, and outer surface may collectively take various shapes depending on the type of use.

In the illustrated implementation of FIG. 3, and as will be described in the more detail below, the geometrical feature 210 is shown as a plurality of boundaries 212 which are each visually distinct from the remainder of the physical volume 204. Each of the plurality of boundaries 212 also can be visually distinct from each of the other boundaries 212. The plurality of boundaries 212 generally includes at least one undercut boundary 214, a target boundary 216, and at least one overcut boundary 218. In the illustrated implementation, the model 200 includes two undercut boundaries 214 and two overcut boundaries 218.

The geometrical feature 210 may be configured to be distinct from the remainder of the physical volume 204, and it is contemplated to create this distinction using any one or more of the following features: a color that is different from a color of the remainder of the physical volume 204, a texture that is different from a texture of the remainder of the physical volume 204, an optical characteristic, such as reflectivity, transparency, and/or opacity, that is different from an optical characteristic of the remainder of the physical volume 204, a density that is different from a density of the remainder of the physical volume 204, and/or a combination thereof. The color, optical, and texture characteristics of the geometrical feature 210 can provide a visible distinction between the geometrical feature 210 and the remainder of the physical volume 204. On the other hand, the density characteristic of the geometrical feature 210 may be arranged to provide a tactile or haptic distinction between the geometrical feature 210 and the remainder of the physical volume 204 as the surgeon/operator removes material from the physical model 200. Other distinctive characteristics/properties are contemplated.

As noted above, the physical model 200 is configured to be physically altered by a surgical instrument 100 (e.g., the handheld surgical instrument 100 (manual or powered) and/or the manipulator 14 (manually or automated)) to provide feedback related to a surgical plan specific to an anatomy of the patient by exposing the geometrical feature 210. In other words, the use of the physical model 200 provides surgeons with preoperative feedback about conducting a surgical plan. By virtue of the physical model 200, the preoperative feedback is physical and tangible. The surgeon can physically experience holding or controlling the cutting tool relative to the physical model 200, thereby providing advantages over computer-implemented surgical simulations. As the surgeon removes portions of the physical volume 204, the geometrical feature 210 is revealed to provide immediate feedback to the surgeon regarding the accuracy of their alteration of the physical volume 204 relative to the surgical plan. If the surgical plan called for a cutting of the bone with a surgical saw, for example, the target boundary 216 would be a plane(s) aligned with the planned cut(s). If the surgeon failed to remove enough of the physical model 200 according to the surgical plan, the undercut boundary 214 may be revealed. Alternatively, if the surgeon removed too much of the physical model 200 according to the surgical plan, the overcut boundary 218 would be revealed. In practice, it is likely that a combination of the undercut boundary(s) 214, target boundary 216, and overcut boundary(s) 218 would be revealed. The surgeon may then determine their accuracy in conducting the surgical plan based on the revealed boundaries 214, 216, 218.

The manipulator 14 may also be programed to conduct the surgical plan on the physical model 200. The accuracy of the surgical robotic system 10 in conducting the surgical plan can be immediately determined based on which of the boundaries 214, 216, 218 are revealed. The surgical system 10 may also include a surface detection system (e.g., including machine vision and/or the localizer 44) which may analyze the remaining surface of the physical model 200 after it has been altered by the instrument 100 or the manipulator 14. The surface detection system may analyze the remaining surface of the physical model 200 if/how the boundaries 214, 216, 218 were cut. This analysis can then be compared to the surgical plan to determine the accuracy of the surgeon/manipulator 14 in conducting the surgical plan. A computing system can implement algorithms for comparing the result of the physical model 200 manipulation relative to the surgical plan. In one example, the surface of the manipulated physical model 200 is mapped using surface detection and the mapped surface is transformed into a coordinate system of a virtual model of the anatomy including the surgical plan. A matching or best-fit algorithm can geometrically align cut surfaces of the physical model 200 (such as portions of the exposed boundaries as obtained from the mapped surface) to the virtual boundaries of the virtual model. Once aligned, the computing system can analyze distances between the cut surfaces the physical model 200 and the virtual boundaries on the virtual model. The computing system can generate an output, such as a report or display indicating precise values of cutting error/accuracy relative to the virtual model boundaries.

The surgical plan may include a planned surface of the patient's tissue. This planned surface may be represented by the target boundary 216. The planned surface may correspond to a surface of an implant meant to be coupled to the patient's tissue after the tissue has been altered by the surgeon/operator. For example, if the surgical plan includes a total knee arthroplasty (TKA), the planned surface of the patient's tissue may be the planned surface of the patient's femur in order to receive a femoral component of a knee implant. The planned surface may correspond to an interior surface of the femoral component which is configured to abut the patient's femur once it has been implanted in the patient.

The physical model 200 may be formed according to various manufacturing techniques using the manufacturing system 90 and the manufacturing machine 94. For example, the physical model 200 may be formed by additive manufacturing, such as 3D printing, heat or laser sintering, laminated object manufacturing, or the like. The physical model 200 may also be formed with various materials or material properties. For example, the physical volume 204 may be made of plastic, thermoplastic, ceramic power, curable resin, polymers, waxes, laminate, or any combination thereof. Further, the physical volume 204 may be formed according to data received by the manufacturing controller 92 from the navigation controller 36, the manipulator controller 26, the tool controller 21, and/or other elements of the control system 60. In one example, the physical volume 204 may be formed with regions of variable density correlating to density information of the anatomy. In such an example, the regions of variable density of the physical volume 204 may correspond to the density of a tissue of the patient as indicated by the navigation controller 36. In another example, and as further described below, the physical volume 204 may be manufactured with various shapes according to plan data received from the navigation controller 36 and/or other elements of the control system 60. If the target of the surgical plan is a human bone, for example, the manufacturing machine 94 may be instructed by the manufacturing system 90 to shape the physical volume 204 similar to the bone. In yet another example, the physical volume 204 may be manufactured with the geometrical feature 210 shaped according to plan data received from the navigation controller 36 and/or other elements of the control system 60. More specifically, the geometrical feature 210 may correspond to plan data including patient imaging data, surgically defined annotations, target boundaries, target trajectories, planned tool paths, locations for any defined feature, thickness of any defined feature, density of volume(s), and/or planned implant sizes/positions, among other plan data.

The body 202 and physical volume 204 of the physical model 200 may be generically shaped (e.g., shaped like a rectangular prism, as shown in FIG. 3) or may be representative of at least a portion of the anatomy of the patient which is a target of the surgical plan (e.g., shaped like a portion of a human bone, as shown in FIG. 4). The undercut boundary 214 may correspond to a section of tissue that should be removed from the patient's tissue according to the surgical plan the target boundary 216 may correspond to a target section of tissue along which the patient's tissue should be cut, and the overcut boundary 218 may correspond to a section of tissue of the patient's tissue that should be avoided by the surgeon/operator.

Referring to FIG. 4, an alternatively shaped body 202 of the physical model 200 is shown. In FIG. 4, the body 202 of the physical model 200 is shaped like the distal end of the patient's femur. The shape of the body 202 may be matched to the target of the surgical plan. For example, if the surgical plan corresponds to a total knee arthroplasty (TKA) procedure, the body 202 may be shaped as shown in FIG. 4. Continuing with the TKA example, the physical model 200 can be coupled to a base 302 representing a femur, the physical model 200 and base 302 can be similar in size, shape, and location of the patient's femur during the surgical procedure as planned. In some implementations, the base 302 is shaped like a generic bone (e.g., femur) and the body 202 is patient specific and shaped like the patient's bone (e.g., distal end of the patient's femur). Alternatively, the body 202 and the base 302 may both be patient specific.

Other shapes are contemplated for the body 202. For example, the body 202 may be shaped like another patient tissue. Hard tissue shapes and soft tissue shapes are contemplated. As described above, where the planned procedure is a TKA, the body 202 may be shaped like a portion of the patient's femur. Similarly, where the planned procedure involves a specific tissue of the patient, the body 202 may be shaped like at least a portion of the specific tissue. As one example of soft tissue, the body 202 may be shaped like a brain of the patient. As will be appreciated based on the above description, the testing system 300 and base 302 may be located and shaped according to the planned procedure. If the planned procedure involves a resection of brain matter, the base 302 may be situated on the operating table near where the head of the patient would be during the planned procedure. The base 302 may be shaped like the bottom half of a brain, and the body 202 may be shaped like the top half of the patient's brain.

Some of the alternative shapes contemplated for the body 202 and physical volume 204 include other hard tissues of the patient. These hard tissues may be specific to the patient's anatomy or may be generalized models. In one example, the physical volume 204 is representative of a proximal and/or distal femur of the patient. In another example, the physical volume 204 is representative of a tibia of the patient. In yet another example, the physical volume 204 is representative of a pelvis and acetabulum of the patient. In yet another example, the physical volume is representative of a scapula and a glenoid of the patient. In yet another example, the physical volume 204 is representative of a proximal humerus of the patient. In yet another example, the physical volume 204 is representative of a vertebra or spine of the patient. In yet another example, the physical volume 204 is representative of a distal femur. In yet another example, the physical volume 204 is representative of a tibia.

Referring back to FIG. 1, the physical model 200 can be a component of a modular system. For example, the physical model 200 may be removably couplable to a testing system 300. More specifically, the testing system 300 may include at least one base 302 which is configured to be removably receive the physical model 200. To that end, the physical model 200 may include a mounting interface 206 arranged to interact with a corresponding mounting interface 306 of the base 302 to removably fix the physical model 200 to the base 302. In the illustrated implementation, the mounting interface 206 is a projection and the corresponding mounting interface 306 is a cavity arranged to receive the projection. Once the physical model 200 is coupled to the base 302, the surgeon/operator can conduct the surgical plan on the physical model 200. After the physical model 200 is altered by the surgeon/operator to expose the geometrical feature 210, the physical model 200 may be removed from the base 302. If, for example, the surgeon/operator would like to try to conduct the surgical plan again, a replacement (unaltered) physical model 200 may be coupled to the base 302. The surgeon/operator may then alter the replacement physical model 200 to determine their accuracy in conducting the surgical plan a second time. Alternatively, the geometrical feature 210 of the replacement physical model 200 may correspond to a different surgical plan to enable the surgeon to determine their accuracy in conducting the different surgical plan. In either case, this process may be repeated by removing the replacement physical model 200 from the base 302 and coupling another replacement physical model 200 to the base 302.

The testing system 300 may be arranged to recreate the environment of an actual patient such that the physical model 200 is located in a similar location of the operating room as the bone to be operated on would be otherwise located during the planned procedure. For example, the testing system 300 shown in FIG. 1 includes two bases 302 which represent the femur and tibia of a patient. These bases 302 may be located where the femur and tibia of the patient would be if the patient were on the operating table. As such, the testing system 300 may include a base support 304 arranged to support the bases 302. The base support 304 may be coupled to or placed on the operating table. Further, the base support 304 may be removably or fixably coupled to the bases 302. The base support 304 may even be in the shape of a model patient with the bones extending therefrom. The testing system 300 may be, but need not necessarily be, located in the operating room. The physical model 200 can be utilized in any environment, including the home of the surgeon.

When the navigation system is utilized, the physical model 200 may further include a registration feature 208 which can register the location of the physical model 200 in the localizer coordinate system. For example, the registration feature 208 may be a divot in the body 202 which is meant to be touched with a surgical probe or observed by the localizer 44. Alternatively, the registration feature 208 can be a detectable tracking feature, such as a QR code, bar code, or reflective/light emitting tracking element. The registration feature 208 may disposed or formed into the physical model 200. Alternatively, the registration feature 208 may include a coupling feature arranged to interact with a tracker. The tracker may then be coupled to the body 202 so that the navigation system 32 may determine and track the location of the body 202 based on the location of the tracker. In such an implementation, the navigation system 32 may know a predefined relationship between the location of the tracker and the location of the body 202.

If the planned procedure is a total knee arthroplasty (TKA), the physical model 200 may represent a distal portion of the patient's femur. As such, the physical model 200 may need to be located near where the distal portion of the patient's femur would be if the patient were in the operating room. The testing system 300 is meant to place the physical model 200 close to where the navigation system 32 would expect the distal portion of the patient's femur to be, and the registration feature 208 provide a precise adjustment to the location of the physical model 200. This can be useful where the manipulator 14 is programmed to conduct the planned procedure on the physical model 200. In order to conduct the planned procedure and to determine the accuracy of the manipulator 14 in carrying out the planned procedure, the navigation system 32 is provided with the precise location of the physical model 200 to know where to carry out the planned cuts/resections.

The base(s) of the testing system 300 may include one or more of the trackers 54, 56 to allow the localizer 44 to determine the location of the base(s) 302. The navigation system 32 may then know an expected position of the physical model 200 based on a known relationship between the trackers 54, 56 and the model 200 once the model 200 is coupled to the base 302. The registration feature 208 then enables confirmation of the actual position of the physical model 200.

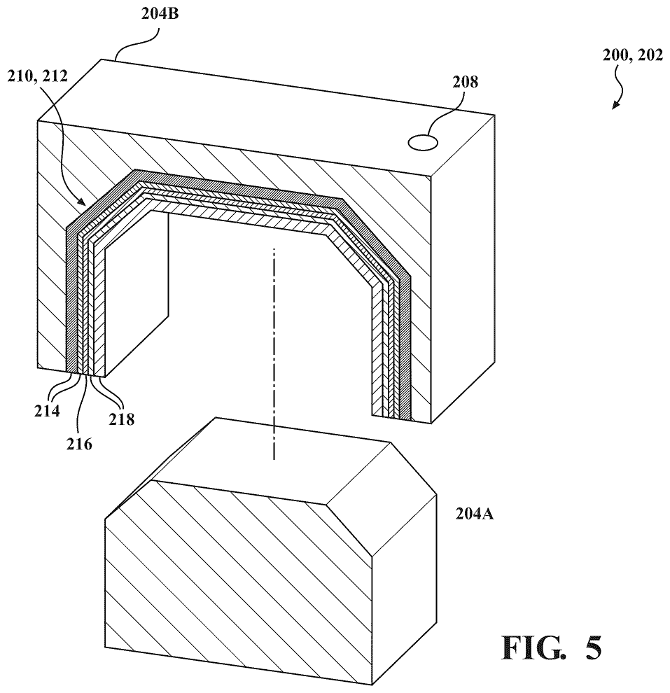

Referring to FIG. 5, an alternative implementation of the modular system is shown. In this example, the physical volume 204 may be split into a first sub-volume 204A and a second sub-volume 204B. In such an implementation, the first sub-volume 204A acts as a receiving base with the second sub-volume 204B configured to be removably attached to the first sub-volume 204A. The second sub-volume 204B may be removably attached to the first sub-volume 204A in any suitable manner and/or via any suitable means. The second sub-volume 204B includes the geometrical feature 210 and, once attached, the second sub-volume 204B is configured to be at least partially removed by the surgical instrument 100 to expose the geometrical feature 210. As in other implementations, the geometrical feature 210 is configured to be exposed to provide visual feedback about an accuracy of the surgical plan and/or an accuracy of the surgical instrument 100 in conducting the surgical plan. Similar to the other modular system implementation, the implementation shown in FIG. 5 allows the surgeon/operator to conduct the surgical plan again by detaching the altered second sub-volume 204B from the first sub-volume 204A and attaching a replacement (unaltered) second sub-volume 204B to the first sub-volume 204A. The surgeon/operator may then alter the replacement second sub-volume 204B to determine their accuracy in conducting the surgical plan a second time. Alternatively, the geometrical feature 210 of the replacement second sub-volume 204B may correspond to a different surgical plan to allow the surgeon to determine their accuracy in conducting the different surgical plan. In either case, this process may be repeated. This modular implementation can be utilized with or without the technique of coupling the physical model 200 to the base 302 like in FIG. 1.

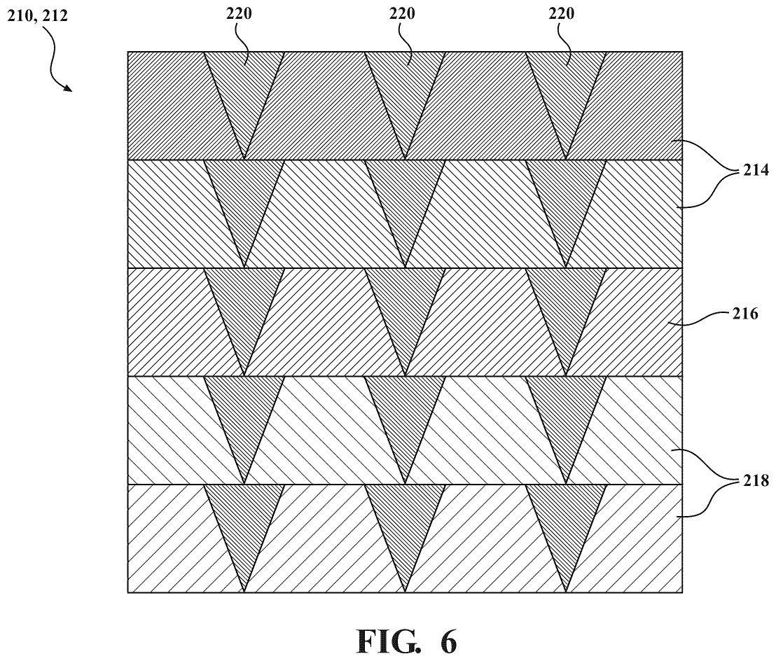

Referring to FIG. 6, a portion of the geometrical feature 210 is shown close up and without the remainder of the physical model 200. In FIG. 6, the geometrical feature 210 includes the boundaries 214, 216, 218 stacked on top of each other. Alternatively, the geometrical feature 210 may be differently shaped/arranged. For example, the geometrical feature 210 may include at least one sub-volume embedded at least partially within the physical volume 204 such that the volume 204 surrounds the sub-volume (not shown). The sub-volumes may be indicative of a planned resection volume of the anatomy that is configured to receive an implant according to the surgical plan, and wherein the planned resection volume has parameters of shape, size and position based on the implant of the surgical plan and a geometry of the anatomy. In such an example, the boundaries 214, 216, 218 may be realized as an undercut resection boundary 214, a target resection boundary 216, and an overcut resection boundary 218, respectively. The undercut resection boundary 214 may be at least partially surrounded by the target resection boundary 216, and the target resection boundary 216 may be at least partially surrounded by the overcut resection boundary 218. In the example shown, the resection boundaries are designed based on the surgical plan defining target resection surfaces for receiving a distal femur implant component at a specific location and orientation relative to the patient anatomy. However, the resection boundaries may be shaped like any three-dimensional shape, such as a sphere, a rectangular prism, and the like. In a more specific example, the target and overcut resection boundaries are bowl-shaped, while the undercut resection boundary is shaped like a half sphere. Other implementations are described herein.

As noted above, the resection volume may have parameters of shape, size and position based on the implant of the surgical plan and a geometry of the anatomy. Further, the body 202 and physical volume 204 may be shaped like the geometry of the anatomy. In one example, the physical volume 204 is representative of a proximal femur of the patient and the planned resection volume comprises a geometry indicative of a target volume of the proximal femur that is configured to receive a femoral stem implant. In another example, the physical volume 204 is representative of a scapula and a glenoid of the patient and the planned resection volume comprises a geometry indicative of a target volume of the glenoid that is configured to receive a glenoid implant. In yet another example, the physical volume 204 is representative of a proximal humerus of the patient and the planned resection volume comprises a geometry indicative of a target volume of the proximal humerus that is configured to receive a humeral stem implant. In yet another example, the physical volume 204 is representative of a vertebra of the patient and the planned resection volume comprises a geometry indicative of a target volume of the vertebra that is configured to receive a pedicle screw. In yet another example, the physical volume 204 is representative of a distal femur of the patient and the planned resection volume comprises a geometry indicative of a target volume of the distal femur that is configured to receive a peg of a femoral implant. In yet another example, the physical volume 204 is representative of a tibia of the patient and the planned resection volume comprises a geometry indicative of a target volume of the tibia that is configured to receive a stem of a tibial implant.

Each of the boundaries 214, 216, 218 comprise a configured certain thickness/depth, for example, of 5-10 millimeters. To that end, the geometrical feature 210 may optionally include depth indicators 220 disposed throughout each of the boundaries 214, 216, 218. In the illustrated implementation, the depth indicators 220 are upside-down solid cones which are visually distinct from the remainder of the physical volume 204 as well as the boundaries 214, 216, 218. As more of a boundary 214, 216, 218 is removed, a smaller cross-section of the cone-shaped depth indicator 220 will remain and present as a circle upon visual inspection of the model 200. More specifically, as more of the boundary 214, 216, 218 is removed, a smaller cross-section of the cone will remain. As an increasing amount of one of the boundaries 214, 216, 218 is removed, the cross-section of the cone will decrease until only a point remains. This provides the surgeon with immediate physical feedback about the depth of the cut while manipulating the physical model 200.

The depth indicators 220 may be different from the illustrated implementation. For example, the depth indicators 220 may be formed of multiple layers of differently color/textured/shaped material. In another example, the depth indicators 220 are a first color at the top of the volume (e.g., green), a second color at the bottom of the volume (e.g., red), and a gradient of colors transitioning from the first color to the second color between the top and bottom of the volume. In another example, the depth indicators 220 are disposed only partially throughout the boundaries 214, 216, 218. More specifically, the depth indicators 220 may be disposed toward the bottoms of each of the boundaries 214, 216, 218. If the boundaries 214, 216, 218 are 5 millimeters thick, the depth indicators 220 may be disposed within the bottom 1 millimeter of each of the boundaries 214, 216, 218. It is also contemplated to include the depth indicators 220 in only some of the boundaries 214, 216, 218. For example, the depth indicators 220 may only be disposed within the target boundary 216.

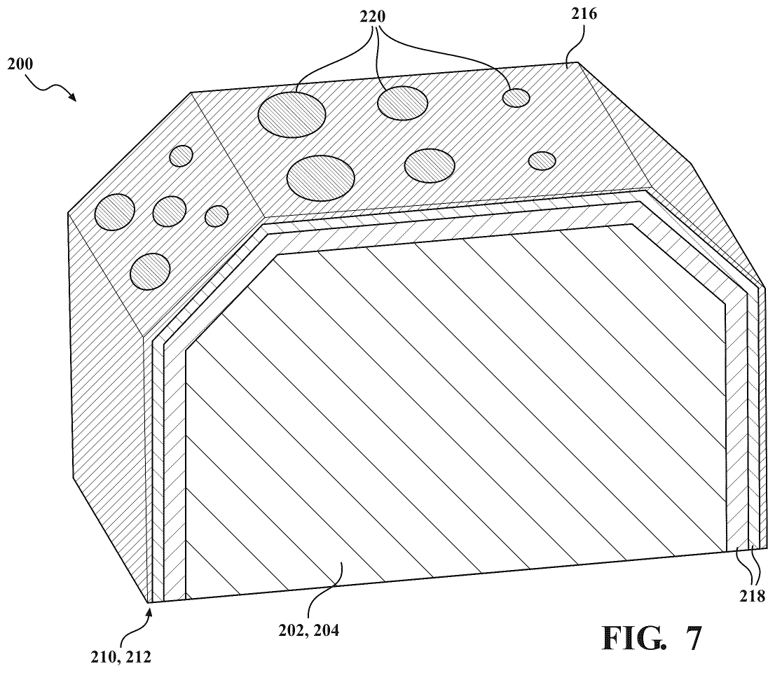

Referring to FIGS. 7-9, example physical models 200 are shown after being cut/resected by the surgeon/operator according to the surgical plan. The models 200 are shown with varying amounts of the geometrical feature 210 being exposed and with varying accuracies. The models 200 are also shown after being cut/resected with different types of instruments. In each of FIGS. 7-9, the body 202 of the physical model 200 is shown with a generic geometry, however, it will be appreciated that the body 202 may instead be shaped like a generic or patient-specific tissue (e.g., the distal end of the patient's femur).

Referring to FIG. 7, the body 202 is shown cut in accordance with the surgical plan with a high degree of accuracy. The geometrical feature 210 has been exposed by the surgeon/operator such that only the target boundary 216 is visible to the surgeon/navigation system 32. Further, circles of varying sizes are shown on the surface of the target boundary 216. These circles represent the depth indicators 220 described above and provide the surgeon/navigation system 32 with insight into how much of the target boundary 216 was removed. As will be appreciated, a certain degree of deviation from the surgical plan may be tolerated. The height of the target boundary 216 may correspond to the tolerated degree of deviation. Although the deviation may be tolerated during the planned procedure, the surgeon/operator may still desire to know how much of the target boundary 216 was removed, and thus how much deviation existed between the surgical plan as planned and the surgical plan as conducted. The physical model 200 of FIG. 7 is meant to illustrate a physical model which was cut/resected accurately with minimal/acceptable deviation.

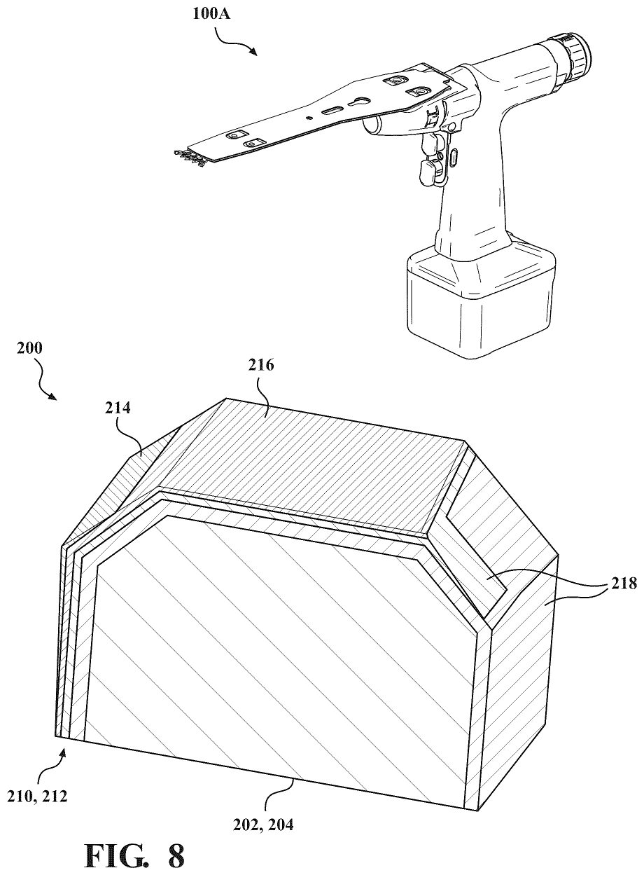

Referring to FIG. 8, the body 202 is shown cut in accordance with the surgical plan with a lower degree of accuracy compared to FIG. 7. As shown in the figure, the body 202 was cut by a handheld surgical saw 100. Less of the physical volume 204 was cut away from the left side of the physical model 200, while more of the physical volume 204 was cut away from the right side of the physical model 200. As such, a portion of the undercut boundary 214 remains present on the left side of the physical model 200, and a portion of the overcut boundary 218 is present on the right side of the physical model 200. Additionally, the top of the physical model 200 was cut in accordance with the surgical plan and the target boundary 216 is therefore present on the top side of the physical model 200. Overall, the surgeon/operator would know that they need to increase the) depth of the cut on the left side and decrease the depth of the cut on the right side.

Referring to FIG. 9, the body 202 is shown resected with a lower degree of accuracy relative to the surgical plan as compared to FIG. 7. As shown in the figure, the body 202 was resected by a handheld surgical instrument 100B which includes a burr. As will be appreciated, surgical saws 100A and surgical burrs 100B cut away patient tissue in different ways. While the surgical saw 100A offers a controlled planar cut, the surgical burr 100B offers a targeted rounded resection. As such, the body 202 of the physical model 200 is shown with varying degrees of each boundary 214, 216, 218 exposed by the burr tool 100B. Based on the varying amounts of the geometrical feature 210 exposed by the burr tool 100B, the surgeon/operator may determine that a differently shaped burr head is appropriate or a change in orientation of the tool is needed for carrying out the surgical plan. Alternatively, the surgeon/operator may determine that the surgical plan should be adjusted. Further, the surgeon/operator may determine that a different tool, such as the surgical saw 100A, would be better suited for the surgical plan.

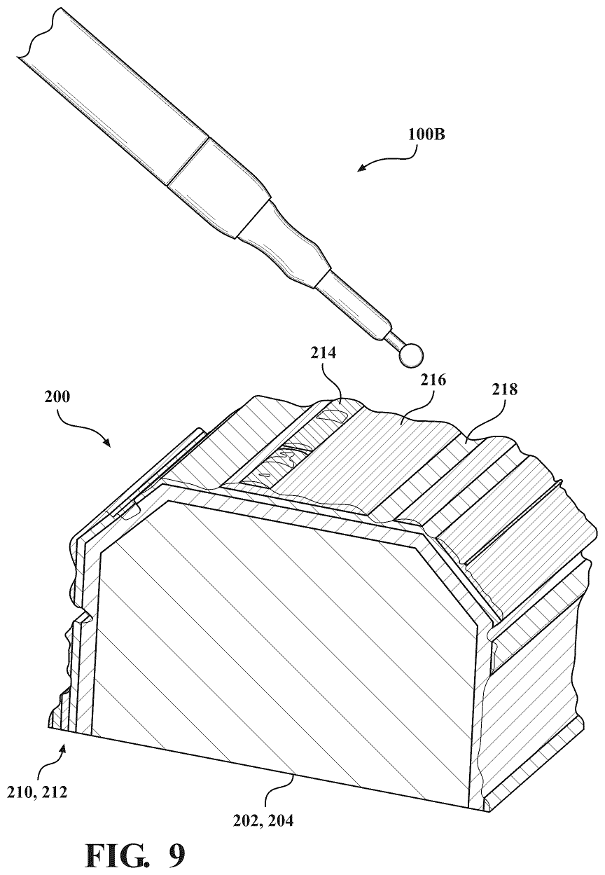

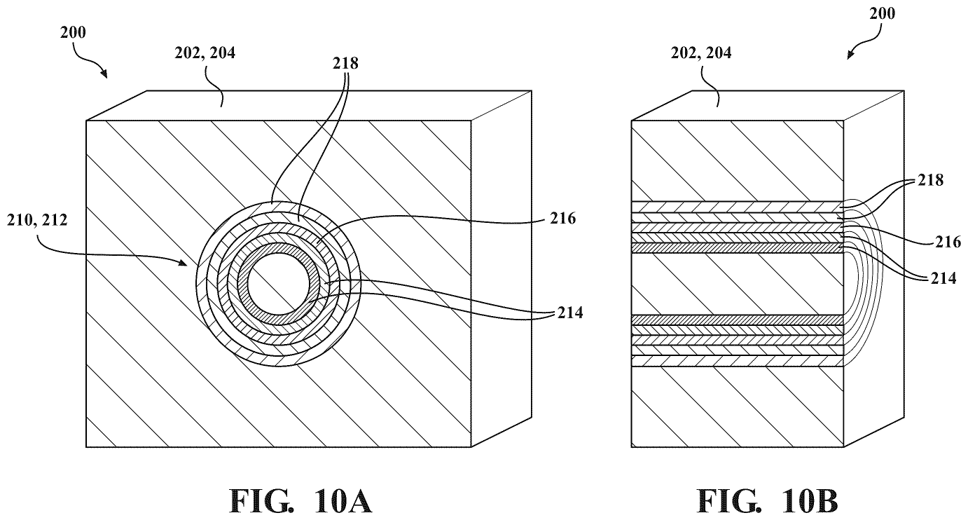

Referring to FIGS. 10A and 10B, another implementation of the physical model 200 is shown. In these figures, the surgical plan generally includes the geometrical feature 210 embodied as a target trajectory relative to the physical model 200. FIG. 10A is a top/side view of the physical model 200, while FIG. 10B is a cross-section of the physical model 200. The geometrical feature 210 of FIGS. 10A and 10B is shown corresponding to the target bore into a patient tissue. For example, the target bore may be a bore arranged to receive a surgical implant, such as a screw. Alternatively, in keeping with the TKA example above, the bore may be a planned bore in a patient's tibia arranged to receive a stem or peg of a component of the knee implant. In any case, the geometrical feature 210 for target trajectories may be shaped as a cylinder with each of the boundaries 214, 216, 218 being cylindrically shaped. The target boundary 216 is realized as a cylinder surrounding the undercut boundary 214 and surrounded by the overcut boundary 218. Both of the undercut and overcut boundaries 214, 218 are also realized as cylinders with radii smaller and larger than the target boundary 216, respectively. The cylinders may be of any desired thickness, for example, 5-10 millimeters thick.

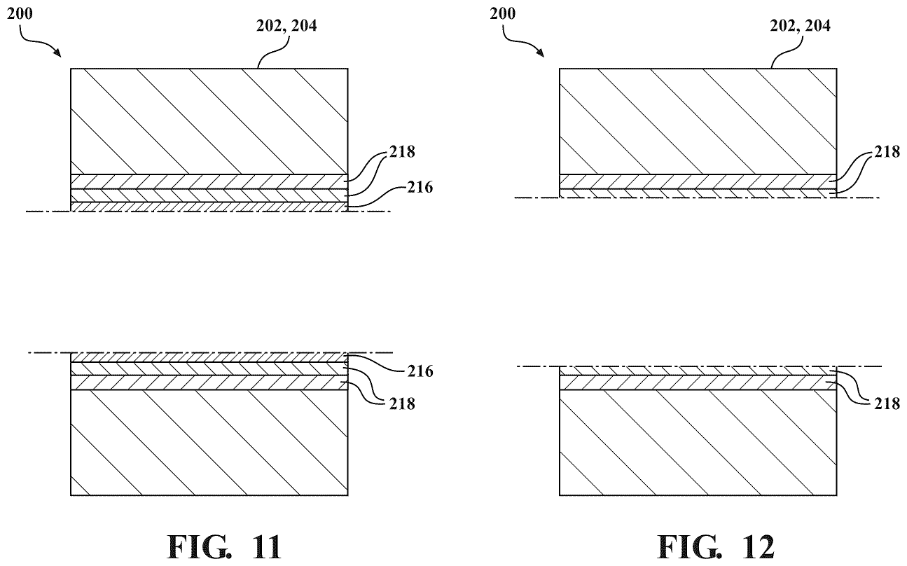

Referring to FIG. 11-15, cross-sectional views of the physical model 200 are shown after the surgeon/operator has drilled through the body 202 of the model 200. FIG. 11 depicts an accurately bored hole according to the surgical plan, while FIG. 12-15 depict inaccurately bored holes with varying causes of this inaccuracy. Similar to the previously described implementations, an accurately bored hole results in only the target boundary 216 being exposed/visible after conducting the planned procedure on the physical model 200. In FIG. 11, the target boundary 216 is exposed, the undercut boundary 216 is removed, and the overcut boundary 218 remains hidden inside of the body 202.

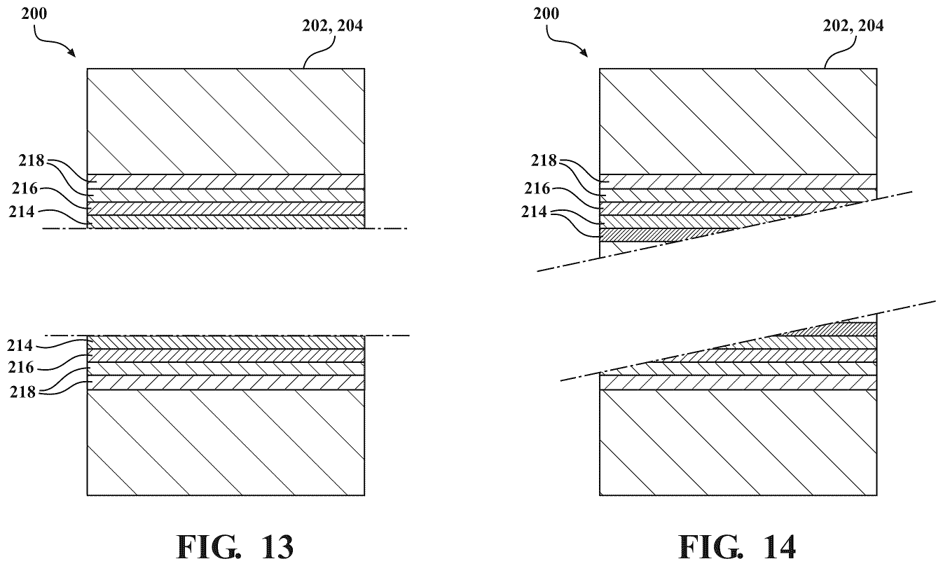

Referring to FIGS. 12 and 13, the body 202 is shown bored with incorrectly sized drill bits. In FIG. 12, the undercut and target boundaries 214, 216 are both shown removed from the body 202 and only the overcut boundaries 218 remain. The surgeon/operator is thus informed that the chosen drill bit was too large/wide by the visibility of the overcut boundary 218. Similarly, in FIG. 13, the undercut boundary 214 is exposed while the target and overcut boundaries 216, 218 remain hidden. The surgeon/operator is thus informed that the chosen drill bit was too small/narrow by the visibility of the undercut boundary 214.

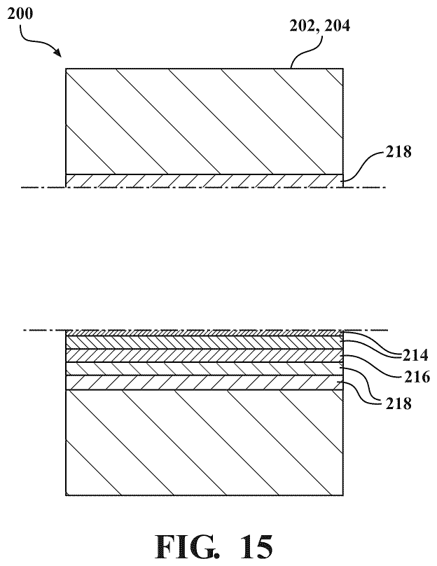

Referring to FIGS. 14 and 15, the body 202 is shown bored along a trajectory out of alignment with the target trajectory. In FIG. 14, the lower portion of the overcut boundary 218 is exposed while the upper portion of the undercut boundary 214 is exposed. The surgeon/operator can receive immediate feedback that a portion of each of the boundaries 214, 216, 218 is visible/exposed along the bore hole. In other words, the surgeon would see a continuously changing spectrum of the boundaries 214, 216, 218 as they looked through the bore hole. The surgeon is thus informed that the orientation of cutting/drilling failed to follow the planned trajectory. Similarly, in FIG. 15, the lower portion of the overcut boundary 218 is exposed and the upper portion of the undercut boundary 214 is exposed. However, the exposed boundaries 214, 218 are consistently visible along the bore hole. As such, the surgeon/operator is informed that the bore followed a path substantially parallel to the target trajectory but spaced away from the target trajectory.

In other cases, the geometrical feature 210 may include a path within the volume 204. For example, the path may include a non-linear/curved path through the volume 204. The path may be indicative of a planned path of the surgical instrument 100 or manipulator 14 relative to the anatomy according to the surgical plan, wherein the planned path has parameters (e.g., position, shape, length, curvature, starting point, ending point, feed rate (tool speed along the path), cutting depth, etc.) based on the surgical plan and the geometry of the anatomy. In one example, the physical volume 204 may be shaped like a bone/tissue of the patient and the path may be indicative of a planned path of the surgical instrument designed to remove a portion from the bone. The planned path may also be indicative of the planned path of the surgical instrument designed to install an implant in the bone. The planned path can be one that the manipulator 14 executes in the manual or automated mode. Deviations from the planned path will be immediately visible to the surgeon after the manipulator 14 is used to modify the physical model 200 according to the planned path.

Although not shown in the figures, the geometrical feature 210 may include the cylindrical boundaries 214, 216, 218 of FIGS. 11-15 as well as planar boundaries disposed normal to the target bore. The planar boundaries 212 may include the undercut, target, and overcut boundaries 214, 216, 218, and these boundaries 214, 216, 218 may correspond to a target depth of the planned bore. Therefore, the surgeon/operator may determine whether the bore hole followed or did not follow the planned trajectory as well as stopped prior to, at, or past the target depth.

In another example, the physical model 200 is representative of an anatomical joint socket, such as an acetabulum or glenoid. The geometrical feature 210 can be implemented into the joint socket to provide feedback about orientation related to reaming in preparation for receiving an implant or related to impaction of the implant. Thus, the surgeon can manipulate the physical model 200 using a reamer and can impact an implant into the physical model 200 using an impactor. During impaction, it is not necessarily required that the surgeon remove material from the physical model 200. In either case, the geometrical feature 210 can be shaped like a cup, or many layered cups, to provide the surgeon with feedback about the orientation of the reaming or impaction relative to the joint socket. For example, the surgical plan may identify the planned version and inclination of socket and the boundaries may be embedded relative to the physical model 200 to provide feedback about whether the surgeon's actions conform to the planned version and inclination or deviate therefrom. For instance, excessive inclined reaming may reveal a sub-layer boundary which is colored red. In the case of impaction, the physical model 200 may include, on a surface layer that is visible to the surgeon, a heat map of colors to indicate the preferred orientation of the cup relative to the joint socket in the physical model 200. For instance, the colors may be implemented as concentric rings of varying colors (e.g., a bullseye). If the implant is impacted in a proper orientation, the implanted implant would reveal a ring indicative of accuracy (e.g., a green ring), while obscuring rings indicative of inaccuracy (e.g., a red ring).

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A physical model configured to be physically altered by a surgical instrument for providing feedback related to a surgical plan specific to an anatomy of a patient, the physical model comprising:
a body including:
a physical volume and a geometrical feature embedded within the physical volume wherein the geometrical feature is visually distinct from a remainder of the physical volume and wherein the geometrical feature has parameters that are based on the surgical plan; and
wherein the physical volume is configured to be at least partially removed by the surgical instrument such that the geometrical feature is exposed to provide visual feedback about an accuracy of the surgical plan and/or an accuracy of the surgical instrument in carrying out the surgical plan.

2. The physical model of claim 1, wherein to be visually distinct from the remainder of the physical volume, the geometrical feature includes a material property that is different from a material property of the remainder of the physical volume.

3. The physical model of claim 1, wherein the physical volume further comprises an outer surface that is shaped to correspond to the anatomy of the patient or shaped according to a generic geometry.

4. The physical model of claim 1, wherein the physical volume and geometrical feature are formed by additive manufacturing.

5. The physical model of claim 1, wherein the physical volume is formed with regions of variable density correlating to density information of the anatomy based on the surgical plan.

6. The physical model of claim 1, wherein the geometrical feature comprises a boundary surface embedded within the physical volume.

7. The physical model of claim 6, wherein the boundary surface is indicative of a region of the anatomy that should be avoided by the surgical instrument according to the surgical plan.

8. The physical model of claim 6, wherein the boundary surface is indicative of a planned resection surface of the anatomy that is configured to receive an implant according to the surgical plan, and wherein the planned resection surface has parameters of shape, size and position based on the implant of the surgical plan and a geometry of the anatomy.

9. The physical model of claim 8, wherein:
the physical volume is representative of a distal femur of the patient and the planned resection surface comprises a plurality of connected planar surfaces indicative of target surfaces of the distal femur that are configured to receive a femoral implant; or
the physical volume is representative of a tibia of the patient and the planned resection surface comprises a planar surface indicative of a target surface of the tibia that is configured to receive a tibial implant; or
the physical volume is representative of a pelvis and acetabulum of the patient and the planned resection surface comprises a concave surface indicative of a target surface of the acetabulum that is configured to receive an acetabular cup implant; or
the physical volume is representative of a scapula and a glenoid of the patient and the planned resection surface comprises a concave surface indicative of a target surface of the glenoid that is configured to receive a glenoid implant.

10. The physical model of claim 6, wherein a 3D indicator having variable cross-sections is embedded within a thickness of the boundary surface, and wherein the boundary surface and the 3D indicator are configured to be at least partially removed by the surgical instrument such that a cross-section of the 3D indicator is configured to be exposed to provide visual feedback related to the thickness of the boundary surface removed or remaining.

11. The physical model of claim 6, wherein the geometrical feature comprises a first boundary surface, a second boundary surface, and a third boundary surface, wherein the first boundary surface is stacked on top of the second boundary surface, and the second boundary surface is stacked on top of the third boundary surface, and wherein each boundary surface is visually distinct from the other boundary surfaces.

12. The physical model of claim 11, wherein the first, second, and third boundary surfaces are configured to be exposed to provide visual feedback related to a cutting accuracy of the surgical instrument, and wherein:
the first boundary surface is configured to be exposed to indicate an undercut;
the second boundary surface is configured to be exposed to indicate an accurate cut; and
the third boundary surface is configured to be exposed to indicate an overcut.

13. The physical model of claim 1, wherein the geometrical feature comprises a sub-volume embedded within and surrounded by the physical volume.

14. The physical model of claim 13, wherein the sub-volume is indicative of a planned resection volume of the anatomy that is configured to receive an implant according to the surgical plan, and wherein the planned resection volume has parameters of shape, size and position based on the implant of the surgical plan and a geometry of the anatomy.

15. The physical model of claim 14, wherein:
the physical volume is representative of a proximal femur of the patient and the planned resection volume comprises a geometry indicative of a target volume of the proximal femur that is configured to receive a femoral stem implant; or
the physical volume is representative of a scapula and a glenoid of the patient and the planned resection volume comprises a geometry indicative of a target volume of the glenoid that is configured to receive a glenoid implant; or
the physical volume is representative of a proximal humerus of the patient and the planned resection volume comprises a geometry indicative of a target volume of the proximal humerus that is configured to receive a humeral stem implant; or
the physical volume is representative of a vertebra of the patient and the planned resection volume comprises a geometry indicative of a target volume of the vertebra that is configured to receive a pedicle screw; or
the physical volume is representative of a distal femur of the patient and the planned resection volume comprises a geometry indicative of a target volume of the distal femur that is configured to receive a peg of a femoral implant; or
the physical volume is representative of a tibia of the patient and the planned resection volume comprises a geometry indicative of a target volume of the tibia that is configured to receive a stem of a tibial implant.

16. The physical model of claim 13, wherein a 3D indicator having variable cross-sections is embedded within a thickness of the sub-volume, and wherein the sub-volume and the 3D indicator are configured to be at least partially removed by the surgical instrument such that a cross-section of the 3D indicator is configured to be exposed to provide visual feedback related to the thickness of the sub-volume removed or remaining.

17. The physical model of claim 13, wherein the geometrical feature comprises a first sub-volume boundary surface, a second sub-volume boundary surface, and a third sub-volume boundary surface, wherein the first sub-volume boundary surface is surrounded by the second sub-volume boundary surface, and the second sub-volume boundary surface is surrounded by the third sub-volume boundary surface, and wherein each sub-volume boundary surface is visually distinct from the other sub-volume boundary surfaces.

18. The physical model of claim 17, wherein the first, second, and third sub-volume boundary surfaces are configured to be exposed to provide visual feedback related to a cutting accuracy of the surgical instrument, and wherein:
the first sub-volume boundary surface is configured to be exposed to indicate an undercut;
the second sub-volume boundary surface is configured to be exposed to indicate an accurate cut; and
the third sub-volume boundary surface is configured to be exposed to indicate an overcut.

19. The physical model of claim 1, wherein the geometrical feature comprises a path embedded within the physical volume.

20. The physical model of claim 19, wherein the path is indicative of a planned path of the surgical instrument relative to the anatomy according to the surgical plan, and wherein the planned path has parameters of shape, length and position based on the surgical plan and a geometry of the anatomy.

21. The physical model of claim 20, wherein the physical volume is representative of a bone of the patient and the path is indicative of the planned path of the surgical instrument designed to remove a portion from the bone.

22. The physical model of claim 20, wherein the physical volume is representative of a bone of the patient and the path is indicative of the planned path of the surgical instrument designed to install an implant in the bone.

23. The physical model of claim 19, wherein the geometrical feature comprises a first path formed as a first cylinder and a second path formed as a second cylinder surrounding the first cylinder, wherein the first path is visually distinct from the second path.

24. The physical model of claim 23, wherein the first and second paths are configured to be exposed to provide visual feedback related to a path accuracy of the surgical instrument, and wherein:
the first path is configured to be exposed to indicate an accurate path of the surgical instrument; and
the second path is configured to be exposed to indicate an inaccurate path of the surgical instrument.

25. The physical model of claim 1, wherein the body further includes a first mounting interface coupled to the body, and the physical model is configured to couple to a base including a second mounting interface arranged to couple with the first mounting interface such that the body is configured to be detachably coupled to the base.

26. The physical model of claim 25, wherein the body is configured to be removed from the base after the physical volume has been at least partially removed by the surgical instrument, and a replacement body substantially similar to the body is configured to be coupled to the base.

27. The physical model of claim 1, wherein the physical volume includes:
a first sub-volume, and
a second sub-volume including the geometrical feature corresponding to the surgical plan and embedded within the second sub-volume; and
wherein the second sub-volume is configured to be attached to the first sub-volume and at least partially removed by the surgical instrument such that the geometrical feature is exposed to provide visual feedback about an accuracy of the surgical plan and/or an accuracy of the surgical instrument in carrying out the surgical plan.

28. The physical model of claim 27, wherein the second sub-volume is configured to be detached from the first sub-volume after the second sub-volume has been at least partially removed by the surgical instrument to facilitate replacement of the second sub-volume.

29. A surgical planning system comprising:
a surgical instrument; and
a physical model configured to be physically altered by the surgical instrument for providing feedback related to a surgical plan specific to an anatomy of a patient, the physical model comprising:
a body including:
a physical volume and a geometrical feature embedded within the physical volume wherein the geometrical feature is visually distinct from a remainder of the physical volume and wherein the geometrical feature has parameters that are based on the surgical plan; and
wherein the physical volume is configured to be at least partially removed by the surgical instrument such that the geometrical feature is configured to be exposed for providing visual feedback about an accuracy of the surgical plan and/or an accuracy of the surgical instrument in carrying out the surgical plan.

30. A method of utilizing a surgical planning system for providing feedback related to a surgical plan specific to an anatomy of a patient, the surgical planning system comprising a surgical instrument and a physical model configured to be physically altered by the surgical instrument, the physical model comprising a body including a physical volume and a geometrical feature embedded within the physical volume wherein the geometrical feature is visually distinct from a remainder of the physical volume and wherein the geometrical feature has parameters that are based on the surgical plan, the method comprising:
utilizing the surgical instrument for at least partially removing the physical volume and for exposing the geometrical feature; and
providing, based on the exposed geometrical feature, visual feedback about an accuracy of the surgical plan and/or an accuracy of the surgical instrument in carrying out the surgical plan.
